(19) United States
(12) Patent Application Publication
(10) Pub. No.: US 2006/0079515 A1
(43) Pub. Date: Apr. 13, 2006

(54) Method for the treatment of polycystic kidney disease

(75) Inventor: Philip Frost, Morris Township, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/243,932

(22) Filed: Oct. 5, 2005

Related U.S. Application Data
(60) Provisional application No. 60/616,981, filed on Oct. 8, 2004.

(51) Int. Cl. A61K 31/5513; A61K 31/537; A61K 31/47; A61K 31/4035; A61K 31/336; A61K 31/19

(52) U.S. Cl. 514/221; 514/227.5; 514/575; 514/313; 514/237.8; 514/475; 514/415

(57) ABSTRACT

The present invention provides a method for treating, inhibiting the progression of, or eradicating polycystic kidney disease of in a patient in need thereof which comprises providing to said patient an effective amount of a TACE inhibitor compound in combination with an effective amount of a Src kinase inhibitor, a HER-2 kinase inhibitor, or a combination of Src and HER-2 inhibitor.

This application claims priority from copending provisional application Ser. No. 60/616,981, filed Oct. 8, 2004, the entire disclosure of which is hereby incorporated by reference.

### FIELD OF INVENTION

The present invention relates to a method of treating polycystic kidney disease. More particularly it involves the use of tumor necrosis factors-alpha converting enzyme (TACE) inhibitor, in combination with other agent(s) such as Src and Human Epidermal Growth Factor Receptor-2 (HER-2) kinase inhibitor, to treat the disease.

### BACKGROUND

Autosomal recessive polycystic kidney disease (ARPKD) is an inherited disorder that usually presents in the newborn period with massive kidney enlargement (due to rapidly expanding cysts) and hepatic fibrosis. ARPKD occurs in approximately 1:10,000 to 1:40,000 births and produces significant morbidity and mortality. Data from experimental models of both recessive and dominant forms of PKD have identified three key pathophysiologic processes in cyst formation and enlargement: increased cell proliferation, increased fluid secretion and altered matrix biology. (Marcia N S, Sweeny W E Armer E D: New insights into the molecular pathophyscology of polycystic kidney disease, Kidney Int., 55:1187-1197, 1999). A growing body of evidence has established the central role of the Src and MEK kinase receptor in the pathogenesis of cell proliferation in PKD.

There is currently no completely effective therapy for polycystic kidney disease. A search for therapeutic agents useful for the treatment of PKD is ongoing.

### SUMMARY OF INVENTION

The present invention provides a method for treating, inhibiting the progression of, or eradicating polycystic kidney disease of in a patient in need thereof which comprises providing to said patient an effective amount of a TACE inhibitor compound in combination with an effective amount of a Src and HER-2 kinase inhibitor alone or in combination.

### DETAILED DESCRIPTION OF THE INVENTION

Preferred TACE inhibitor compounds are described in WO 00/44730, WO 00/44749, WO 00/44709, WO 00/44711, WO 00/44710, WO 00/44716, WO 00/44740, WO 00/44713, and WO 00/44723 each of which is hereby incorporated by reference thereto.

Especially preferred TACE inhibitor compounds include those of formula I 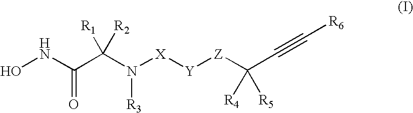
wherein:
X is SO₂ or —P(O)—R₁₀;
Y is aryl or heteroaryl, with the proviso that X and Z may not be bonded to adjacent atoms of Y;
Z is O, NH, CH₂ or S;
R₁ is hydrogen, aryl, alkyl of 1-6 carbon atoms, alkenyl of 2-6 carbon atoms, alkynyl of 2-6 carbon atoms;
R₂ is hydrogen, aryl, aralkyl, heteroaryl, heteroaralkyl, cycloalkyl of 3-6 carbon atoms, C₄-C₈ cycloheteroalkyl, alkyl of 1-6 carbon atoms, alkenyl of 2-6 carbon atoms, alkynyl of 2-6 carbon atoms;
or R₁ and R₂, together with the atom to which they are attached, may form a ring wherein R₁ and R₂ represent a divalent moiety of the formula: 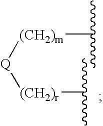
wherein
   Q=a carbon-carbon single or double bond, O, S, SO, SO₂, —N—R₁₁, or —CONR₁₄;
   m=1-3;
   r=1 or 2, with the proviso that when Q is a bond, r is equal to 2;
R₃ is hydrogen, alkyl of 1-6 carbon atoms, cycloalkyl of 3-6 carbon atoms, C4-C8 cycloheteroalkyl, aralkyl, or heteroaralkyl;
or R₁ and R₃, together with the atoms to which they are attached, may form a 5 to 8 membered ring wherein R₁ and R₃ represent divalent moieties of the formulae: 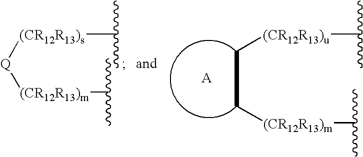
wherein Q and m are as defined above;
A is aryl or heteroaryl;
s is 0-3;
u is 1-4;
R₄ and R₅ are each, independently, hydrogen or alkyl of 1-6 carbon atoms, —CN, or —CCH;
R₆ is hydrogen, aryl, heteroaryl, alkyl of 1-6 carbon atoms, alkenyl of 2-6 carbon atoms, alkynyl of 2-6 carbon atoms, cycloalkyl of 3-6 carbon atoms, or —C₅-C₈-cycloheteroalkyl;
R₈ and R₉ are each, independently, hydrogen, alkyl of 1-6 carbon atoms, alkenyl of 2-6 carbon atoms, alkynyl of 2-6 carbon atoms, cycloalkyl of 3-6 carbon atoms, aryl, aralkyl, heteroaryl, heteroaralkyl, or —C₄-C₈-cycloheteroalkyl;
R₁₀ is alkyl of 1-6 carbon atoms, cycloalkyl of 3-6 carbon atoms, aryl or heteroaryl;
R₁₁ is hydrogen, alkyl of 1-6 carbon atoms, cycloalkyl of 3-6 carbon atoms, aryl, heteroaryl, —S(O)ₙR₈, —COOR₈, —CONR₈R₉, —SO₂NR₈R₉ or —COR₈;
R₁₂ and R₁₃ are independently selected from H, —OR₈, —NR₈R₉, alkyl of 1-6 carbon atoms, alkenyl of 2-6 carbon atoms, alkynyl of 2-6 carbon toms, cycloalkyl of 3-6 carbon atoms, aryl, heteroaryl, —COOR₈; —CONR₈R₉; or R₁₂ and R₁₃ together form a —C₃-C₆-cycloalkyl of 3-6 carbon atoms or a —C₅-C₈-cycloheteroalkyl ring; or R₁₂ and R₁₃, together with the carbon to which they are attached, form a carbonyl group;
with the proviso that R₁₀ and R₁₂ or R₁₁ and R₁₂ may form a cycloheteroalkyl ring when they are attached to adjacent atoms;
R₁₄ is hydrogen, aryl, heteroaryl, alkyl of 1-6 carbon atoms or cycloalkyl of 3-6 carbon atoms;
and n is 0-2;
   or a pharmaceutically acceptable salt thereof.

Heteroaryl, as used throughout, is a 5-10 membered mono- or bicyclic ring having from 1-3 heteroatoms selected from N, NR₁₄, S and O. Heteroaryl is preferably an aromatic ring selected from 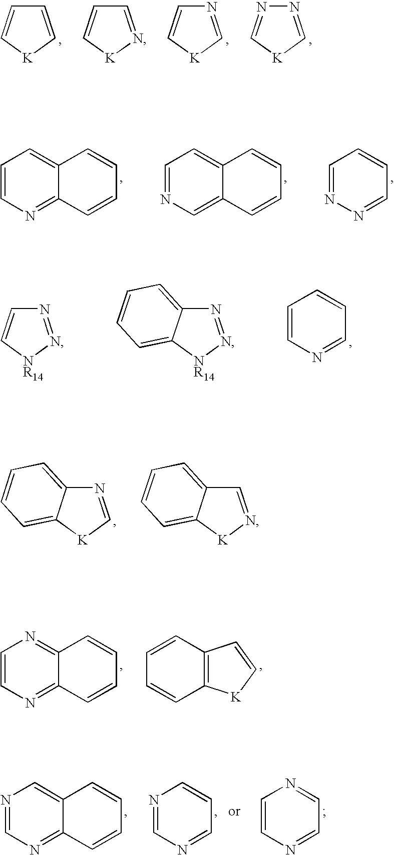
wherein K is O, S or —NR₁₄ and R₁₄ is hydrogen, aryl, heteroaryl, alkyl of 1-6 carbon atoms, or cycloalkyl of 3-6 carbon atoms. Preferred heteroaryl rings include pyrrole, furan, thiophene, pyridine, pyrimidine, pyridazine, pyrazine, triazole, pyrazole, imidazole, isothiazole, thiazole, isoxazole, oxazole, indole, isoindole, benzofuran, benzothiophene, quinoline, isoquinoline, quinoxaline, quinazoline, benzotriazole, indazole, benzimidazole, benzothiazole, benzisoxazole, and benzoxazole. Heteroaryl groups may optionally be mono or di substituted.

Cycloheteroalkyl as used herein refers to a 5 to 9 membered saturated or unsaturated mono or bi-cyclic ring having 1 or 2 heteroatoms selected from N, NR₁₄, S or O. Heterocycloalkyl rings of the present invention are preferably selected from; 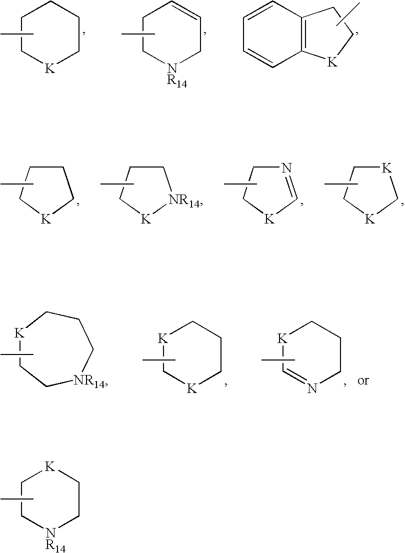
wherein K is NR₁₄, O or S and R₁₄ is a bond, hydrogen, aryl, heteroaryl, alkyl of 1-6 carbon atoms, or cycloalkyl of 3-6 carbon atoms.

Cycloheteroalkyl as used herein refers to a 5 to 9 membered saturated or unsaturated mono or bi-cyclic ring having 1 or 2 heteroatoms selected from N, NR₁₄, S or O. Heterocycloalkyl rings of the present invention are preferably selected from; 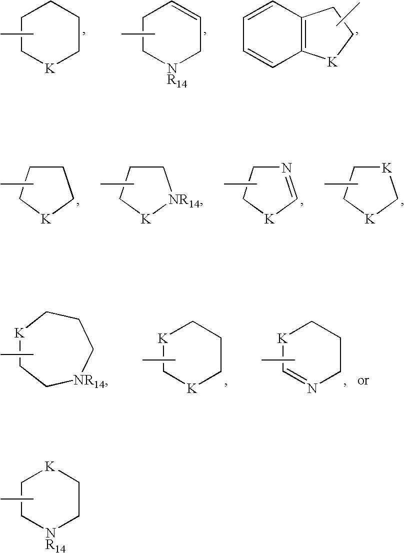
wherein K is NR₁₄, O or S and R₁₄ is a bond, hydrogen, aryl, heteroaryl, alkyl of 1-6 carbon atoms, or cycloalkyl of 3-6 carbon atoms.

Preferred heterocycloalkyl rings include piperidine, piperazine, morpholine, tetrahydropyran, tetrahydrofuran or pyrrolidine. Cycloheteroalkyl groups of the present invention may optionally be mono- or di- substituted.

Aryl, as used herein refers to a phenyl or napthyl rings which may, optionally be mono-, di- or tri-substituted.

Alkyl, alkenyl, alkynyl, and perfluoroalkyl include both straight chain as well as branched moieties. Alkyl, alkenyl, alkynyl, and cycloalkyl groups may be unsubstituted (carbons bonded to hydrogen, or other carbons in the chain or ring) or may be mono- or poly-substituted. Lower alkyl moieties contain from 1 to 6 carbon atoms.

Aralkyl as used herein refers to a substituted alkyl group, -alkyl-aryl, wherein alkyl is lower alkyl and preferably from 1 to 3 carbon atoms, and aryl is as previously defined.

Heteroaralkyl as used herein refers to a substituted alkyl group, alkyl-heteroaryl wherein alkyl is lower alkyl and preferably from 1 to 3 carbon atoms, and heteroaryl is as previously defined.

Halogen means bromine, chlorine, fluorine, and iodine.

Suitable substituents of aryl, aralkyl, heteroaryl, heteroaralkyl, alkyl, alkenyl, alkynyl, and cycloalkyl include, but are not limited to hydrogen, halogen, alkyl of 1-6 carbon atoms; alkenyl of 2-6 carbon atoms; alkynyl of 2-6 carbon atoms, cycloalkyl of 3-6 carbon atoms, —OR₈, —[[O(CH₂)ₚ]_{q}]—OCH3, CN, —COR₈, perfluoroalkyl of 1-4 carbon atoms, —O-perfluoroalkyl of 1-4 carbon atoms, —CONR₈R₉, —S(O)ₙR₈, —S(O)ₙR₁₈C(O)OR₈, —S(O)ₙR₁₈OR₉, —S(O)ₙR₁₈NR₈R₉, —S(O)ₙR₁₈NR₈R₉COOR₈, —S(O)ₙR₁₈NR₈COR₉, —OPO(OR₈)OR₉, —PO(OR₈)R₉, —OC(O)NR₈R₉, —C(O)NR₈OR₉, —C(O)R₁₈NR₈R₉, —COOR₈, —SO₃H, —NR₈R₉, —N[(CH₂)₂]₂NR₈, —NR₈COR₉, —NR₈C(O)CH═CHaryl, —NR₈C(O)(CH₂)ₙNR₈R₉, —NR₈C(O)CH₂NHCH₂aryl, NR₈C(O)R₁₈, —NR₈COOR₉, —SO₂NR₈R₉, —NO₂, —N(R₈)SO₂R₉, —NR₈CONR₈R₉, —NR₈C(═NR₉)NR₈R₉, —NR₈C(═NR₉)N(SO2R₈)R₉, NR₈C(═NR₉)N(C═OR₈)R₉-tetrazol-5-yl, —SO₂NHCN, —SO₂NHCONR₈R₉, —(OR18)NR₈S(O)R₉, —(OR₁₈)NR₈C(O)R₉, —(OR₁₈)NR₈C(O)NR₈R₉, —(OR18)NR₈COOR₉, —(OR₁₈)NR₈R₉, phenyl, heteroaryl, or C₄-C₈-cycloheteroalkyl;
wherein —NR₈R₉ may form a heterocyclic group as previously defined, such as pyrrolidine, piperidine, morpholine, thiomorpholine, oxazolidine, thiazolidine, pyrazolidine, piperazine, and azetidine ring; p is 1 or 2,
q is 1 through 3 and
R₁₈ is alkyl of 1-20 carbon atoms.

In some preferred embodiments of the present invention R₈ and R₁₈ may be further substituted with halogen, C₁-C₃ alkyl, C₁-C₃ alkoxy and OH, and NO₂.

When a moiety contains more than substituent with the same designation (i.e., phenyl tri-substituted with R₁) each of those substituents (R₁ in this case) may be the same or different.

TACE inhibitor compounds of the present invention include compounds of formula II, III and IV: 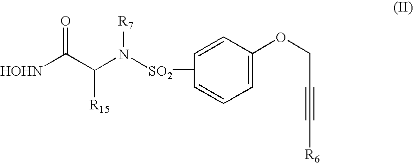
wherein
R₆ is as defined above with CH₃ and CH₂OH being preferred; R₇ is H or alkyl with H or methyl being preferred; and R₁₅ is alkyl, with isopropyl and CH(CH₃)OH being preferred. 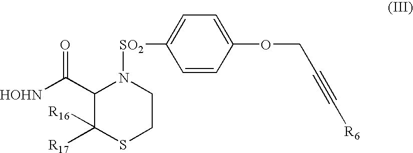
wherein R₆ is defined as above with methyl and CH₂OH being preferred;
R₁₆ and R₁₇ are alkyl preferably methyl. 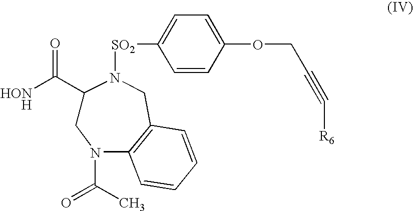
wherein R₆ is as defined above with methyl being preferred.

TACE inhibitor compounds include 4-(4-but-2-ynyloxy-benzenesulfonyl)-2,2-dimethyl-thiomorpholine-3-carboxylic acid hydroxyamide; (3S)—N-hydroxy-4-({4-[(4-hydroxy-2-butynyl)oxy]phenyl}sulfonyl)-2,2-dimethyl-3-thiomorpholinecarboxamide; (2R)—N-hydroxy-2-[({4-[(4-hydroxy-2-butynyl)oxy]phenyl}sulfonyl)(methyl)amino]-3-methylbutanamide; and (2R,3S)-2-({[4-(2-butynyloxy)phenyl]sulfonyl}amino)-N,3-dihydroxybutanamide.

The present invention also encompasses a method for the treatment of ARPKD by using a TACE inhibitors compound in combination with an Src or HER-2 receptor kinase inhibitor alone or in combination wherein the Src inhibitor includes compounds of the formula: 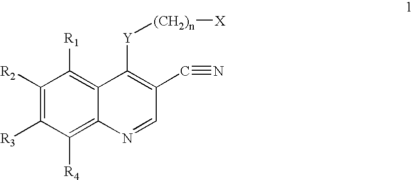
wherein:
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
n is 0-1;
Y is —NH—, —O—, —S—, or —NR—;
R is alkyl of 1-6 carbon atoms;
R₁, R₂, R₃, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 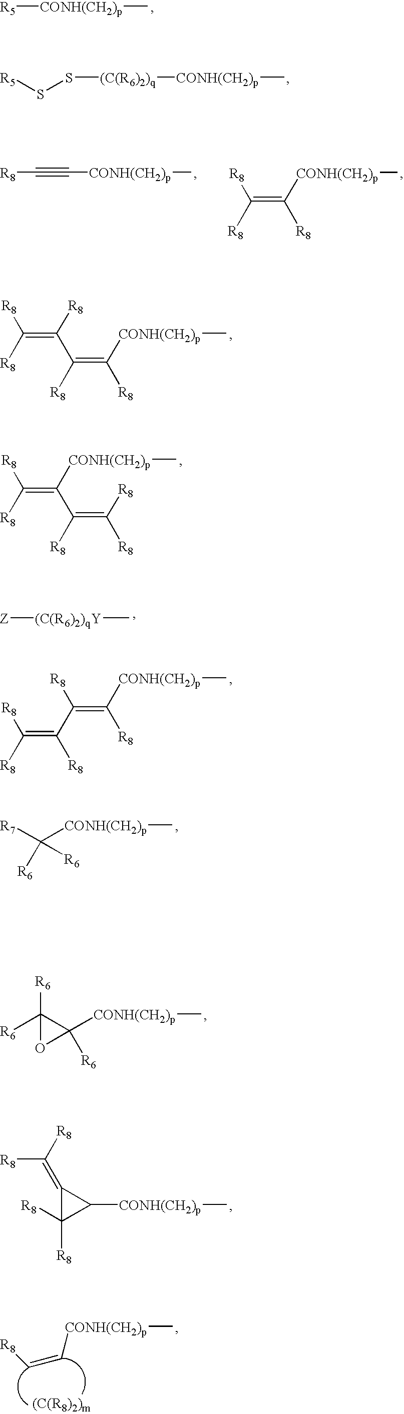 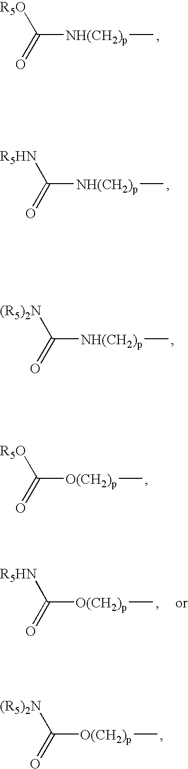
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo
R₈ is hydrogen, alkyl of 1-6 carbon atoms, aminoalkyl of 1-6 cabon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
Z is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;
m=1-4, q=1-3, and p=0-3;
any of the substituents R₁, R₂, R₃, or R₄ that are located on contiguous carbon atoms can together be the divalent radical —O—C(R₈)₂—O—;
or a pharmaceutically acceptable salt thereof with the proviso that when Y is —NH—, R₁, R₂, R₃, and R₄ are hydrogen, and n is 0, X is not 2-methylphenyl:

Preferred Src receptor kinase inhibitor compounds are described in U.S. Pat. No. 6,002,008 which compounds are hereby incorporated by reference. The compound 4-(2,4-dichloro-5-methoxy-phenylamino)-6-methoxy-7-[3-(4-methyl-piperazin-1-yl)-propoxy]-quinoline-3-carbonitrile is especially preferred.

Preferred HER-2 receptor kinase inhibitor compounds are described in U.S. Pat. No. 6,288,082 which compounds are hereby incorporated by reference.

The present invention includes HER-2 receptor kinase inhibitors having the structure: 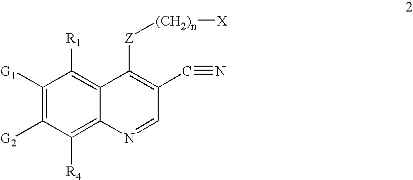
wherein:
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino; or
X is a radical radical having the formula: 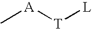
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon ctoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino;
T is bonded to a carbon of A and is:
   —NH(CH₂)ₘ—, —O(CH₂)ₘ—, —S(CH₂)ₘ—, —NR(CH₂)ₘ—, —(CH₂)ₘ—
   —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino; provided that L can be an unsubstituted phenyl ring only when m>0 and T is not —CH₂NH— or —CH₂O—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino;
Z is —NH—, —O—, —S—, or —NR—;
R is alkyl of 1-6 carbon atoms, or carboalkyl of 2-7 carbon atoms;
G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 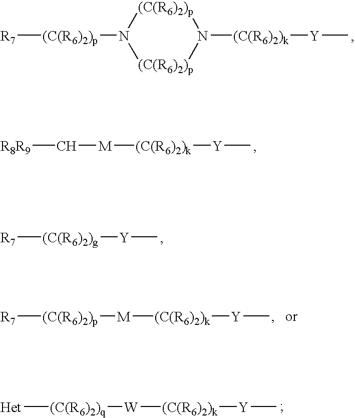
or R₁ and R₄ are as defined above and G₁ or G₂ or both are R₂—NH—; or if any of the substituents R₁, G₂, G₃, or R₄ are located on contiguous carbon atoms then they may be taken together as the divalent radical —O—C(R₆)₂—O—;
Y is a divalent radical selected from the group consisting of 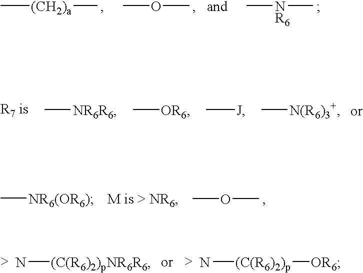
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 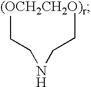
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)_{S}OR₆ or —(C(R₆)₂)_{S}N(R₆)₂, and optionally mono or di-substituted on a saturated carbon with divalent radicals —O— or —O(C(R₆)₂)_{S}O—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of: 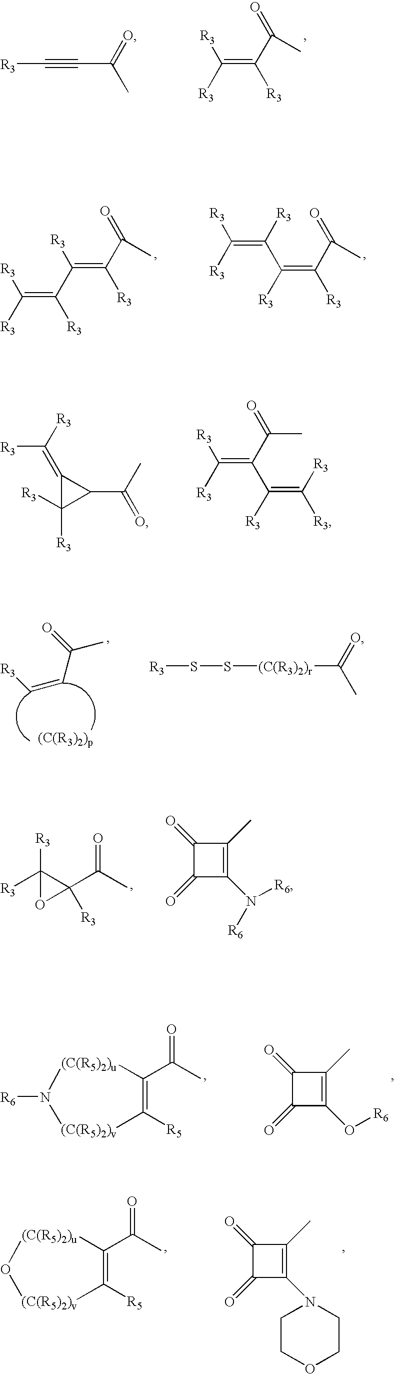 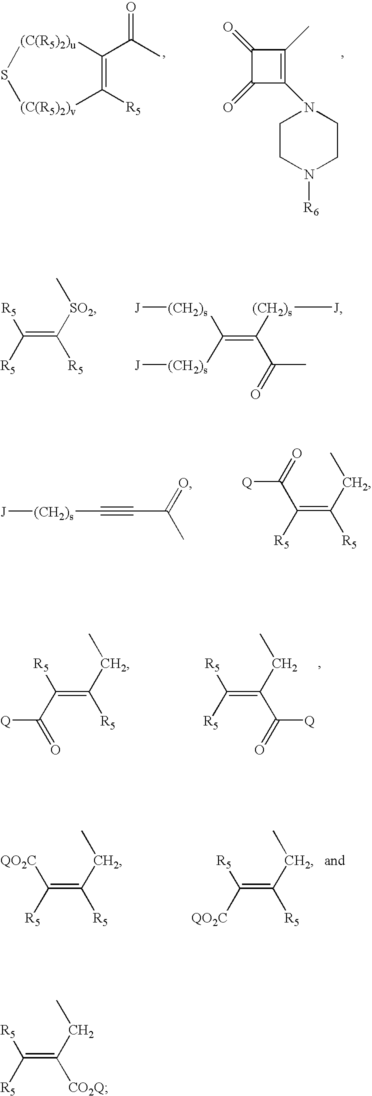
R₃ is independently hydrogen, alkyl of 1-6 carbon atoms, carboxy, carboalkoxy of 1-6 carbon atoms, phenyl, carboalkyl of 2-7 carbon atoms, 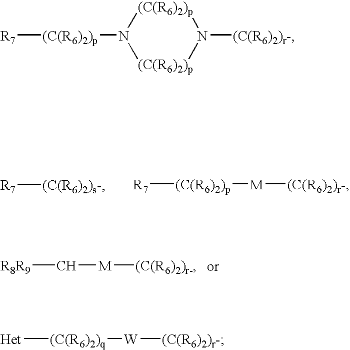
R₅ is independently hydrogen, alkyl of 1-6 carbon atoms, carboxy, carboalkoxy of 1-6 carbon atoms, phenyl, carboalkyl of 2-7 carbon atoms, 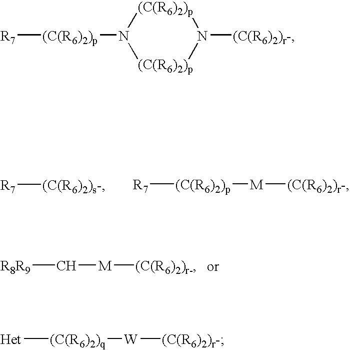
R₈, and R₉ are each, independently, —(C(R₆)₂)ᵣNR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4;
or a pharmaceutically acceptable salt thereof,
provided that
   when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that
   when Y is —NR₆— and R₇ is —NR₆R₆, —N(R₆)₃⁺, or —NR₆(OR₆), then g=2-6;
   when M is —O— and R₇ is —OR₆ then p=1-4;
   when Y is —NR₆— then k=2-4;
   when Y is —O— and M or W is —O— then k=1-4
   when W is not a bond with Het bonded through a nitrogen atom then q=2-4
and when W is a bond with Het bonded through a nitrogen atom and Y is, —O— or —NR₆— then k=2-4.

HER-2 receptor kinase inhibitor compounds also include compounds having the formula: 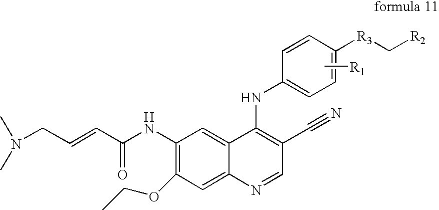
wherein:
R₁ is halogen;
R₂ is a pyridinyl, thiophene, optionally substituted pyrimidine, or an optionally substituted phenyl ring wherein the phenyl or pyrimidine ring may be unsubstituted, mono-substituted, or di-substituted; and
R₃ is —O— or —S—.

Preferred HER-2 receptor kinase inhibitor compounds include (E)-N-{4-[3-chloro-4-(2-pyridinyl methoxy)anilino]-3-cyano-7-ethoxy-6-quinolinyl}-4-(dimethylamino)-2-butenamide, (E)-N-{4-[4-(benzyloxy)-3-chloroanilino]-3-cyano-7-ethoxy-6-quinolinyl}-4-(dimethylamino)-2-butenamide, (E)-N-{4-[3-chloro-4-(2-pyridinylmethoxy)anilino]-3-cyano-7-ethoxy-6-quinolinyl}-4-(dimethylamino)-2-butenamide, (E)-N-(4-{3-chloro-4-[(3-fluorobenzyl)oxy]anilino}-3-cyano-7-ethoxy-6-quinolinyl)-4-(dimethylamino)-2-butenamide, (2E)-N-(4-{[3-chloro-4-(1,3-thiazol-2-ylsulfanyl)phenyl]amino}-3-cyano-7-methoxy-6-quinolinyl)-4-(dimethylamino)-2-butenamide, (E)-N-(4-{3-chloro-4-[(4,6-di-methyl-2-pyrimidinyl)sulfanyl]anilino}-3-cyano-7-ethoxy-6-quinolinyl)-4-(dimethylamino)-2-butenamide, a compound comprising (E)-N-{4-[3-chloro-4-(1,3-thiazol-2-ylsulfanyl)anilino]-3-cyano-7-methoxy-6-quinolinyl}-4-[(2-methoxyethyl)(methyl)amino]-2-butenamide or a pharmaceutically acceptable salt thereof.

In the present invention “an effective amount” of the Src or HER-2 receptor kinase inhibitor compound will vary with inter alia the individual patient and the severity of the disease, however generally it will be at least about 5 mg/kg. A preferred range is about 10 to 50 mg/kg.

In the present invention “an effective amount” of the TACE inhibitor compound will vary with a variety of factors including the individual patient and the severity of the disease. Typically the effective amount will be at least about 5 mg/kg. A preferred range is about 20 to 40 mg/kg.

The dosing schedule of the drug(s) may be from once to several times per day or may be less frequent. Preferably the dosing will be less frequent, for example dosing every other day, every third day or once a week.

In the present invention, the terms TACE inhibitor, TACE inhibitor compound, EGFR receptor kinase inhibitor, and EGFR receptor kinase inhibitor compound include all optical isomers and diastereomers as well as pharmaceutically acceptable salts.

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains an acidic moiety.

The compounds of this invention may contain an asymmetric carbon atom and some of the compounds of this invention may contain one or more asymmetric centers and may thus give rise to optical isomers and diastereomers. While shown without respect to stereochemistry, the present invention includes such optical isomers and diastereomers; as well as the racemic and resolved, enantiomerically pure R and S stereoisomers; as well as other mixtures of the R and S stereoisomers and pharmaceutically acceptable salts thereof. It is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

An effective amount of the compound[s] of the invention are provided to the patient. The compounds may be provided orally, in liquid or solid form, or by injection. In addition the compound may be provided to the patient via a pro-drug route wherein the patient actually converts in vivo a substance given to him or her to one or more of the TACE inhibitors or EGFR receptor kinase inhibitors of the present invention.

The following examples are merely illustrative of the present invention. The invention is not to be limited thereby.

### EXAMPLE 1

TACE inhibitor alone or in combination with a Src inhibitor, a HER-2 inhibitor, or a combination Src inhibitor and HER-2 inhibitor or a combination of Src inhibitor and HER-2 inhibitor was studied in vitro on primary collecting tubule (CT) cells from human polycystic kidney disease (PKD) samples, control and cystic primary CT cells derived from the rat homologue of human PKD and control and cystic conditionally immortalized CT cells from the BPK mouse model of PKD. A showing of decreasing cyst development and growth is presented. Additional information is presented with respect to compound toxicity, cellular proliferation, site specific phosphorylation levels of EGFR, c-Src, MEK and downstream targets.

### EXAMPLE 2

TACE inhibitor alone or in combination with a Src inhibitor, a HER-2 inhibitor, or a combination Src inhibitor and HER-2, or Src inhibitor and HER-2 inhibitor in combination inhibitor was studied in vivo in the PCK rat (the rat homologue of human PKD) to determine the effectiveness of the compounds alone or in combination on ameliorating the progression of both renal and hepatic cyst development and growth. Initial studies were conducted on 3 control and 3 cystic rats using doses ranging from 10 to 60 mg/kg daily and every third day. Beginning at postnatal day 7, a Src inhibitor was administered IP at varying concentrations and frequency until postnatal day 28. Survival, renal and hepatic function, morphometric analysis (cyst size and number), as well as site specific phosphorylation levels of upstream and downstream targets of c-Src were assessed. When the dose that provides the maximum effect with minimum toxicity was determined, 10 control and 10 cystic animals were treated for a minimum of 10 weeks. A complete necropsy was performed on both a normal and cystic animal.

What is claimed is:

1. A method for treating, inhibiting the progression of, or eradicating polycystic kidney disease in a mammal in need thereof which comprises providing to said mammal an effective amount of a TACE inhibitor compound in combination with a Src inhibitor, a HER-2 inhibitor or a combination of Src inhibitor and HER-2 inhibitor.

2. The method according to claim 1 wherein the TACE inhibitor is a compound of formula I: 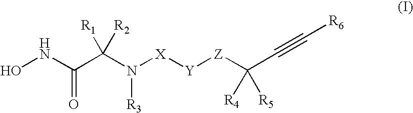
wherein:
X is SO₂ or —P(O)—R₁₀;
Y is aryl or heteroaryl, with the proviso that X and Z may not be bonded to adjacent atoms of Y;
Z is O, NH, CH₂ or S;
R₁ is hydrogen, aryl, alkyl of 1-6 carbon atoms, alkenyl of 2-6 carbon atoms, alkynyl of 2-6 carbon atoms;
R₂ is hydrogen, aryl, aralkyl, heteroaryl, heteroaralkyl, cycloalkyl of 3-6 carbon atoms, C₄-C₈ cycloheteroalkyl, alkyl of 1-6 carbon atoms, alkenyl of 2-6 carbon atoms, alkynyl of 2-6 carbon atoms;
or R₁ and R₂, together with the atom to which they are attached, may form a ring wherein R₁ and R₂ represent a divalent moiety of the formula: 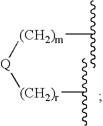
wherein
Q=a carbon-carbon single or double bond, O, S, SO, SO₂, —N—R₁₁, or —CONR₁₄;
m=1-3;
r=1 or 2, with the proviso that when Q is a bond, r is equal to 2;
R₃ is hydrogen, alkyl of 1-6 carbon atoms, cycloalkyl of 3-6 carbon atoms, C₄-C₈ cycloheteroalkyl, aralkyl, or heteroaralkyl;
or R₁ and R₃, together with the atoms to which they are attached, may form a 5 to 8 membered ring wherein R₁ and R₃ represent divalent moieties of the formulae: 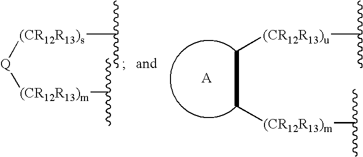
wherein Q and m are as defined above;
A is aryl or heteroaryl;
s is 0-3;
u is 1-4;
R₄ and R₅ are each, independently, hydrogen or alkyl of 1-6 carbon atoms, —CN, or —CCH;
R₆ is hydrogen, aryl, heteroaryl, alkyl of 1-6 carbon atoms, alkenyl of 2-6 carbon atoms, alkynyl of 2-6 carbon atoms, cycloalkyl of 3-6 carbon atoms, or —C₅-C₈-cycloheteroalkyl;
R₈ and R₉ are each, independently, hydrogen, alkyl of 1-6 carbon atoms, alkenyl of 2-6 carbon atoms, alkynyl of 2-6 carbon atoms, cycloalkyl of 3-6 carbon atoms, aryl, aralkyl, heteroaryl, heteroaralkyl, or —C₄-C₈-cycloheteroalkyl;
R₁₀ is alkyl of 1-6 carbon atoms, cycloalkyl of 3-6 carbon atoms, aryl or heteroaryl;
R₁₁ is hydrogen, alkyl of 1-6 carbon atoms, cycloalkyl of 3-6 carbon atoms, aryl, heteroaryl, —S(O)ₙR₈, —COOR₈, —CONR₈R₉, —SO₂NR₈R₉ or —COR₈;
R₁₂ and R₁₃ are independently selected from H, —OR₈, —NR₈R₉, alkyl of 1-6 carbon atoms, alkenyl of 2-6 carbon atoms, alkynyl of 2-6 carbon atoms, cycloalkyl of 3-6 carbon atoms, aryl, heteroaryl, —COOR₈; —CONR₈R₉; or R₁₂ and R₁₃ together form a —C₃-C₆-cycloalkyl of 3-6 carbon atoms or a —C₅-C₈-cycloheteroalkyl ring; or R₁₂ and R₁₃, together with the carbon to which they are attached, form a carbonyl group;
with the proviso that R₁₀ and R₁₂ or R₁₁ and R₁₂ may form a cycloheteroalkyl ring when they are attached to adjacent atoms;
R₁₄ is hydrogen, aryl, heteroaryl, alkyl of 1-6 carbon atoms or cycloalkyl of 3-6 carbon atoms;
and n is 0-2;
or a pharmaceutically acceptable salt thereof.

3. The method according to claim 2 wherein the compound is a compound of formula II: 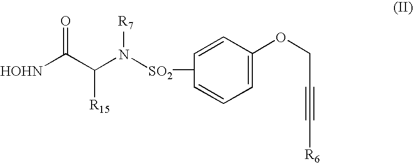
wherein
R₆ is as defined in claim 2; R₇ is H or alkyl; and R₁₅ is alkyl.

4. The method according to claim 3 wherein R₆ is CH₃ or CH₂OH; R₇ is H or methyl; and R₁₅ is isopropyl or CH(CH₃)OH.

5. The method according to claim 2 wherein the compound is a compound of formula III: 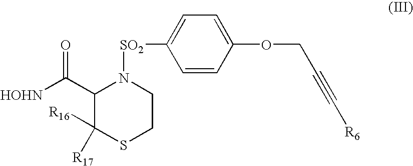
wherein R₆ is defined as in claim 2 with methyl and CH₂OH being preferred;
and R₁₆ and R₁₇ are alkyl preferably methyl.

6. The method according to claim 5 wherein R₆ is methyl or CH₂OH; and R₁₆ and R₁₇ are methyl.

7. The method according to claim 1 wherein said mammal is given an effective amount of a TACE inhibitor compound of formula IV: 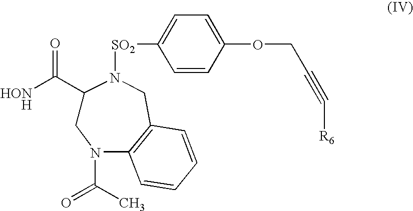
wherein R₆ is as defined in claim 3.

8. The method according to claim 7 wherein R₆ is methyl.

9. The method according to claim 1 wherein the Src kinase inhibitor is a compound having the formula 1: 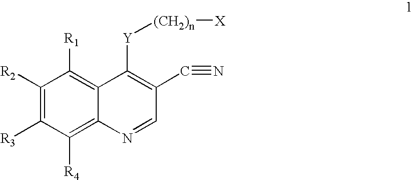
wherein:
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
n is 0-1;
Y is —NH—, —O—, —S—, or —NR—;
R is alkyl of 1-6 carbon atoms;
R₁, R₂, R₃, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 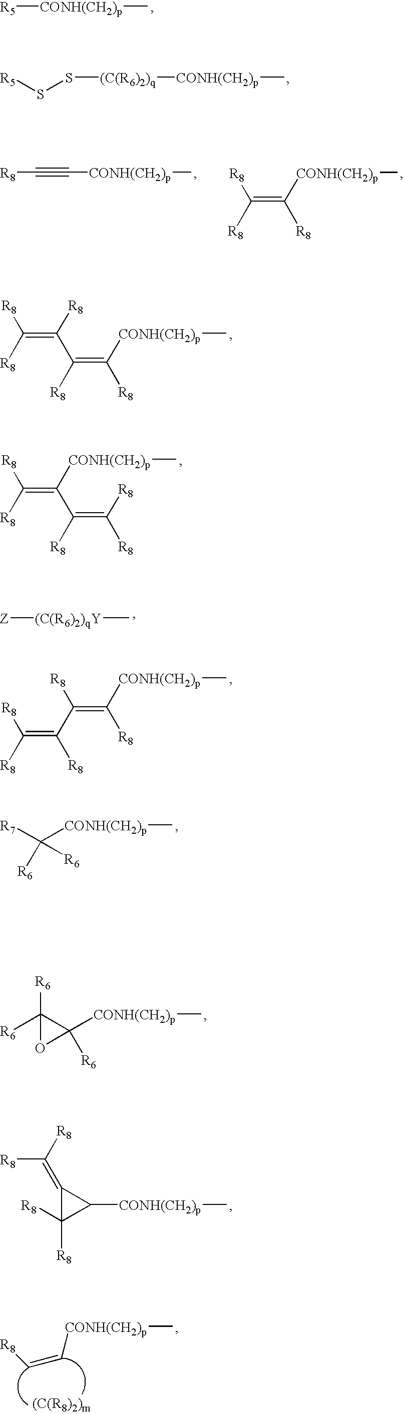 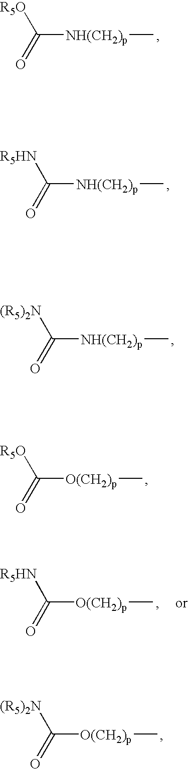
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo;
R₈ is hydrogen, alkyl of 1-6 carbon atoms, aminoalkyl of 1-6 cabon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
Z is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;
m=1-4, q=1-3, and p=0-3;
any of the substituents R₁, R₂, R₃, or R₄ that are located on contiguous carbon atoms can together be the divalent radical —O—C(R₈)₂—O—;
or a pharmaceutically acceptable salt thereof with the proviso that when Y is —NH—, R₁, R₂, R₃, and R₄ are hydrogen, and when n is 0, X is not 2-methylphenyl and R₃ is not Cl.

10. The method according to claim 9 wherein the Src kinase inhibitor is 4-(2,4-dichloro-5-methoxy-phenylamino)-6-methoxy-7-[3-(4-methyl-piperazin-1-yl)-propoxy]-quinoline-3-carbonitrile.

11. The method according to claim 1 wherein the HER-2 inhibitor is a compound of formula 2 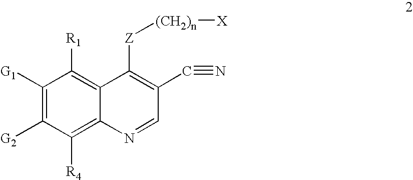
wherein:
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino; or
X is a radical radical having the formula: 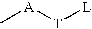
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino;
T is bonded to a carbon of A and is:
—NH(CH₂)ₘ—, —O(CH₂)ₘ—, —S(CH₂)ₘ—, —NR(CH₂)ₘ—, —(CH₂)ₘ—
—(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino; provided that L can be an unsubstituted phenyl ring only when m>0 and T is not —CH₂NH— or —CH₂O—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalky of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, methylmercapto, and benzoylamino;
Z is —NH—, —O—, —S—, or —NR—;
R is alkyl of 1-6 carbon atoms, or carboalkyl of 2-7 carbon atoms;
G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 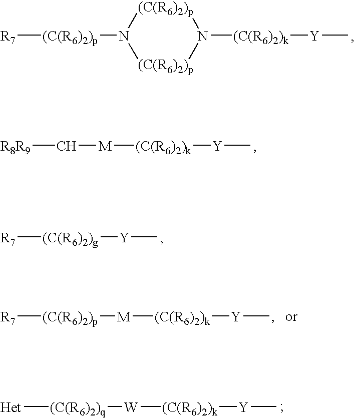
or R₁ and R₄ are as defined above and G₁ or G₂ or both are R₂—NH—;
or if any of the substituents R₁, G₂, G₃, or R₄ are located on contiguous carbon atoms then they may be taken together as the divalent radical —O—C(R₆)₂—O—;
Y is a divalent radical selected from the group consisting of 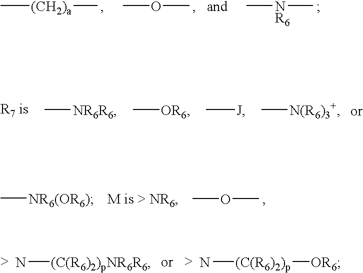
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 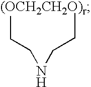
wherein Het is optionally mono- or di-substituted on carbon or nitrogen with R₆, optionally mono- or di-substituted on carbon with hydroxy, —N(R₆)₂, or —OR₆, optionally mono or di-substituted on carbon with the mono-valent radicals —(C(R₆)₂)_{S}OR₆ or —(C(R₆)₂)_{S}N(R₆)₂, and optionally mono or di-substituted on a saturated carbon with divalent radicals —O— or —O(C(R₆)₂)_{S}O—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 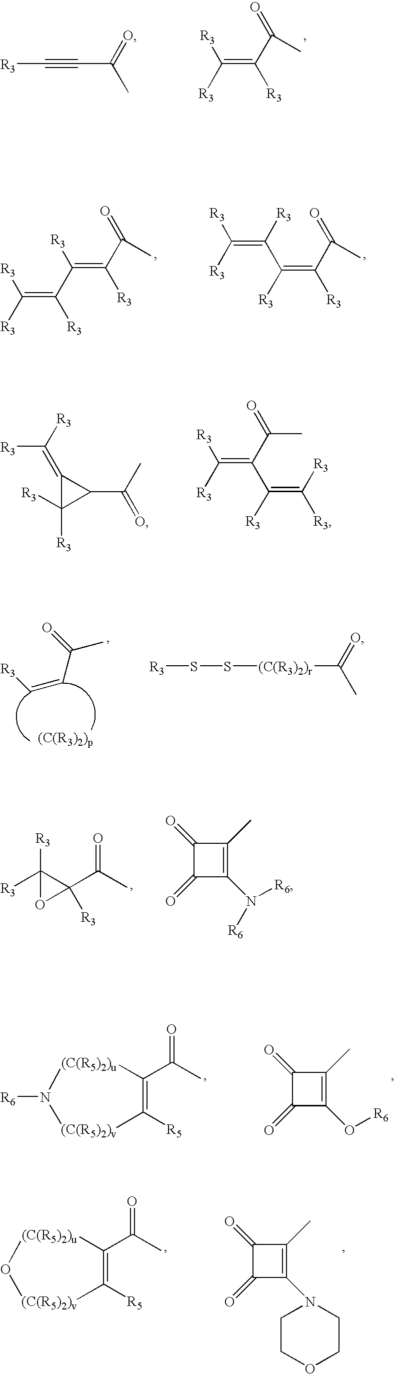 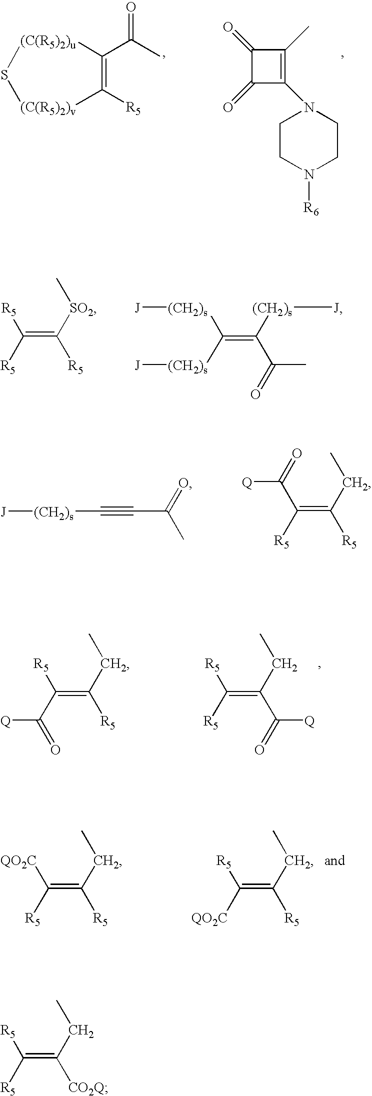
R₃ is independently hydrogen, alkyl of 1-6 carbon atoms, carboxy, carboalkoxy of 1-6 carbon atoms, phenyl, carboalkyl of 2-7 carbon atoms, 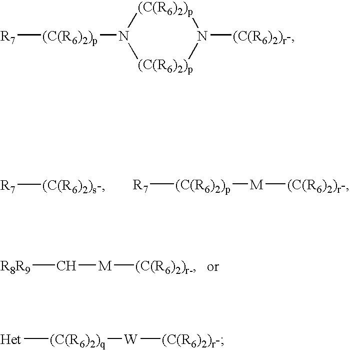
R₅ is independently hydrogen, alkyl of 1-6 carbon atoms, carboxy, carboalkoxy of 1-6 carbon atoms, phenyl, carboalkyl of 2-7 carbon atoms, 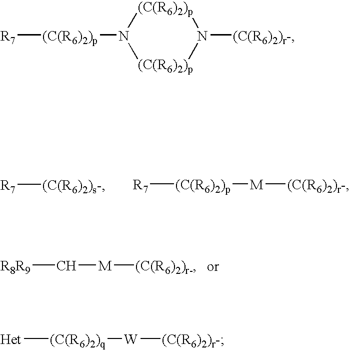
R₈, and R₉ are each, independently, —(C(R₆)₂)ᵣNR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4;
or a pharmaceutically acceptable salt thereof,
provided that
when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that
when Y is —NR₆— and R₇ is —NR₆R₆, —N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4
when W is not a bond with Het bonded through a nitrogen atom then q=2-4
and when W is a bond with Het bonded through a nitrogen atom and Y is —O— or —NR₆— then k=2-4.

12. The method according to claim 11 wherein the HER-2 inhibitor is a compound of formula: 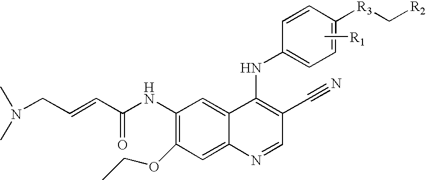
wherein:
R₁ is halogen;
R₂ is a pyridinyl, thiophene, optionally substituted pyrimidine, or an optionally substituted phenyl ring wherein the phenyl or pyrimidine ring may be unsubstituted, mono-substituted, or di-substituted; and
R₃ is —O— or —S—.

13. The compound according to claim 12, in which the HER-2 inhibitor is:
(E)-N-{4-[4-(benzyloxy)-3-chloroanilino]-3-cyano-7-ethoxy-6-quinolinyl}-4-(dimethylamino)-2-butenamide or a pharmaceutically acceptable salt thereof.

14. The compound according to claim 12, in which the HER-2 inhibitor is:
(E)-N-(4-[3-chloro-4-(2-pyridinylmethoxy)anilino]-3-cyano-7-ethoxy-6-quinolinyl}-4-(dimethylamino)-2-butenamide or a pharmaceutically acceptable salt thereof.

15. The compound according to claim 12, in which the HER-2 inhibitor is:
(E)-N-(4-{3-chloro-4-[(3-fluorobenzyl)oxy]anilino}-3-cyano-7-ethoxy-6-quinolinyl)-4-(dimethylamino)-2-butenamide or a pharmaceutically acceptable salt thereof.

16. The compound according to claim 12, in which the HER-2 inhibitor is:
(2E)-N-(4-{[3-chloro-4-(1,3-thiazol-2-ylsulfanyl)phenyl]amino}-3-cyano-7-methoxy-6-quinolinyl)-4-(dimethylamino)-2-butenamide or a pharmaceutically acceptable salt thereof.

17. The compound according to claim 12, in which the HER-2 inhibitor is:
(E)-N-(4-{3-chloro-4-[(4,6-di-methyl-2-pyrimidinyl)sulfanyl]anilino)-3-cyano-7-ethoxy-6-quinolinyl)-4-(dimethylamino)-2-butenamide or a pharmaceutically acceptable salt thereof.

18. The compound of claim 12, in which the HER-2 inhibitor is:
(E)-N-{4-[3-chloro-4-(1,3-thiazol-2-ylsulfanyl)anilino]-3-cyano-7-methoxy-6-quinolinyl}-4-[(2-methoxyethyl)(methyl)amino]-2-butenamide or a pharmaceutically acceptable salt thereof.

19. The method according to claim 12 wherein the HER-2 inhibitor is (E)-N-{4-[3-chloro-4-(2-pyridinyl methoxy)anilino]-3-cyano-7-ethoxy-6-quinolinyl}-4-(dimethylamino)-2-butenamide.

18. A method for treating, inhibiting the progression of, or eradicating polycystic kidney disease in a mammal in need thereof which comprises providing to said mammal an effective amount of a combination of a Src inhibiting compound and a HER-2 inhibiting compound.

19. A method for treating, inhibiting the progression of, or eradicating polycystic kidney disease in a mammal in need thereof which comprises providing to said mammal an effective amount of a Src inhibiting compound.

20. A method for treating, inhibiting the progression of, or eradicating polycystic kidney disease in a mammal in need thereof which comprises providing to said mammal an effective amount of a HER-2 inhibiting compound.

21. A method for treating, inhibiting the progression of, or eradicating polycystic kidney disease in a mammal in need thereof which comprises providing to said mammal an effective amount of a Src inhibiting compound.

22. A method for treating, inhibiting the progression of, or eradicating polycystic kidney disease in a mammal in need thereof which comprises providing to said mammal an effective amount of a HER-2 inhibiting compound.

23. A product comprising a TACE inhibitor compound and (i) a Src inhibitor or (ii) a HER-2 inhibitor; or (iii) a Src inhibitor and a HER-2 inhibitor
as a combined preparation for simultaneous, separate or sequential use in treating, inhibiting the progression of, or eradicating polycystic kidney disease in a mammal.

24. A pharmaceutical composition for treating, inhibiting the progression, or eradicating polycystic kidney disease in a mammal comprising a TACE inhibitor compound and (i) a Src inhibitor or (ii) a HER-2 inhibitor; or (iii) a Src inhibitor and a HER-2 inhibitor, and a pharmaceutically acceptable carrier.
